(19) United States
(12) Patent Application Publication
(10) Pub. No.: US 2009/0214469 A1
(43) Pub. Date: Aug. 27, 2009

(54) Methods and Compositions for the Treatment of Sepsis

(76) Inventors: Jeffrey Gold, Portland, OR (US); James J. Lee, Scottsdale, AZ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/392,673

(22) Filed: Feb. 25, 2009

Related U.S. Application Data
(60) Provisional application No. 61/031,258, filed on Feb. 25, 2008.

(51) Int. Cl. A61K 38/20

(52) U.S. Cl. 424/85.2

(57) ABSTRACT

Methods for the treatment of sepsis are disclosed.

This application claims priority under 35 U.S.C. §119(e) to U.S. Provisional Patent Application No. 61/031,258, filed on Feb. 25, 2008. The foregoing application is incorporated by reference herein.

Pursuant to 35 U.S.C. §202(c), it is acknowledged that the U.S. Government has certain rights in the invention described herein, which was made in part with funds from the National Institutes of Health, Grant Nos. HL065228 and HL058723.

### FIELD OF THE INVENTION

The present invention relates to the fields of immunology and pharmacology. More specifically, the invention provides methods for the treatment of sepsis and septic shock.

### BACKGROUND OF THE INVENTION

Several publications and patent documents are cited throughout the specification in order to describe the state of the art to which this invention pertains. Each of these citations is incorporated herein by reference as though set forth in full.

Sepsis, the systemic inflammatory response to infection, affects over 700,000 people/year in the United States with nearly 250,000 deaths (Hotchkiss et al. (2003) N. Engl. J. Med., 348:138-50; Angus et al. (2001) Crit. Care Med., 29:1303-10). This costs $17 billion/year and is the leading cause of death in ICU and the 10th leading cause of death overall. Despite recent advances and adequate antimicrobial therapy, mortality remains in excess of 25% (Hotchkiss et al. (2003) N. Engl. J. Med., 348:138-50; Angus et al. (2001) Crit. Care Med., 29:1303-10).

In attempts to improve mortality, multiple studies have focused on the attenuation of the initial cytokine storm present in sepsis and septic shock. The host inflammatory response in sepsis is extremely complex involving an intricate interplay between pro and anti-inflammatory mediators with divergent effects on pathogen control, systemic inflammation and susceptibility to recurrent infection. Numerous attempts to alter the balance of inflammatory cytokine production in sepsis through modulation individual cytokines have been largely unsuccessful. Neutralization of pro-inflammatory cytokines such as IL-1β and TNF-α fails to improve outcome in humans and mice with polymicrobial sepsis (Opal et al. (1997) Crit. Care Med., 25:1115-24; Remick et al. (1998) Crit. Care Med., 26:895-904; Remick et al. (1995) Shock 4:89-95; Abraham et al. (1998) Lancet 351:929-33; Cohen et al. (1996) Crit. Care Med., 24:1431-40). These strategies failed in part due to the redundant and overlapping function of many inflammatory cytokines, making neutralization of individual cytokines ineffective. In addition, by altering the balance between pro and anti-inflammatory mediators, these strategies led to impairment in host defense and pathogen control. Consequently, investigators have focused on upstream receptors/mediators which are capable of regulating numerous inflammatory cascades (Echtenacher et al. (2001) Infect. Immun., 69:7271-6; Qureshi et al. (1999) J. Exp. Med., 189:615-25; Ebong et al. (2001) Infect. Immun., 69:2099-106; Weighardt et al. (2002) J. Immunol., 169:2823-7; Watanakunakorn et al. (1991) Scand. J. Infect. Dis., 23:399-405).

The ability of pathogen recognition receptors to control numerous inflammatory cascades led investigators to attempt blockade of these molecules in order to improve mortality in sepsis. However, Toll Like Receptor 4^{-/-} (TLR4) mice, or mice treated with either α-TLR4 or α-CD14 antibodies, while protected in endotoxemia, have either unchanged or increased mortality in models of polymicrobial sepsis such as Cecal Ligation and Puncture (CLP) (Echtenacher et al. (2001) Infect. Immun., 69:7271-6; Qureshi et al. (1999) J. Exp. Med., 189:615-25; Ebong et al. (2001) Infect. Immun., 69:2099-106; Weighardt et al. (2002) J. Immunol., 169:2823-7). Similar results were obtained with TLR2^{-/-} mice (Qureshi et al. (1999) J. Exp. Med., 189:615-25). Together this suggests inhibition of the primary mechanism of pathogen recognition may result in a damaging degree of innate immune impairment and subsequent failure of pathogen control.

These studies have renewed interest in better understanding of host cells involved in pathogen control. Monocytes and neutrophils (polymorphonuclear cells; PMNs) are essential for the host control of infection and it is well established that the presence of leukocytopenia and/or granulocytopenia is an independent risk factor for mortality in patients with pneumonia or sepsis (Watanakunakorn et al. (1991) Scand. J. Infect. Dis., 23:399-405; Weinstein et al. (1983) Rev. Infect. Dis., 5:54-70; Georges et al. (1999) Intensive Care Med., 25:198-206). Furthermore, in non-neutropenic individuals, sepsis is associated with immunoparalysis and/or deactivation of innate immune effector cells. This is supported by the reduction in HLA-DR expression on circulating monocytes and the impairment in ex vivo bacterial killing by PMNs from septic individuals (Solomkin et al. (1985) Arch. Surg., 120:93-8; Zimmerman et al. (1989) Crit. Care Med., 17:1241-6; Docke et al. (1997) Nat. Med., 3:678-81; Heumann et al. (1998) Curr. Opin. Infect. Dis., 11:279-83). Based on these observations, studies have begun to focus on growth and activation factors responsible for granulocyte maturation and function. The best studied of these is GM-CSF. GM-CSF administration, while resulting in recruitment of additional and potentially pro-inflammatory leukocytes, improves pathogen control and bacterial clearance in mice and humans (Rosenbloom et al. (2005) Chest 127:2139-50; Orozco et al. (2006) Arch. Surg., 141:150-154; Presneill et al. (2002) Am. J. Respir. Crit. Care Med., 166:138-43). In parallel experiments, studies have focused on the administration of leukocyte derived granule proteins and defensins to augment the host defense in an attempt to compensate for reduced leukocyte activity (Motzkus et al. (2006) Faseb J., 20:1701-2). One example is Bactericidal Permeability Inhibitor (BPI). A major component of PMN granules, BPI is capable of binding endotoxin as well as possessing a de novo antibacterial activity against gram⁺ and gram⁻ bacteria (Ooi et al. (1991) J. Exp. Med., 174:649-55; Gazzano-Santoro et al. (1992) Infect. Immun., 60:4754-61; Weiss et al. (1992) J. Clin. Invest., 90:1122-30). Recently, administration of recombinant BPI significantly improved morbidity (amputations) and showed a trend towards improved mortality in children with meningococcemia (Levin et al. (2000) Lancet 356:961-7).

### SUMMARY OF THE INVENTION

In accordance with the present invention, compositions and methods for treating sepsis and/or septic shock or reducing the risk of developing sepsis and/or septic shock in a patient are provided. The methods comprise administering an effective amount composition comprising IL-5 and at least one pharmaceutically acceptable carrier. In a specific embodiment of the instant invention, the method further comprises that administration of at least one other anti-sepsis agent.

### BRIEF DESCRIPTION OF THE DRAWINGS

FIG. 1 is a graph demonstrating that IL-5 transgenic mice have improved survival after cecal ligation and puncture (CLP). Wild-type (WT) and NJ1638^{±} mice underwent CLP with an 18 gauge needle and monitored for survival for 14 days. No deaths occurred after day 5.

FIG. 2 provides graphs demonstrating IL-5 transgenic mice have improved bacterial clearance after CLP. WT and NJ1638 mice underwent CLP. Blood (FIG. 2A) and peritoneal lavage (PL; FIG. 2B) were harvested for quantitative cultures. N=6/group. Data was analyzed by nonparametric T-Test.

FIG. 3 provides graphs demonstrating IL-5 transgenic mice have little change in inflammatory cytokines after CLP. WT and NJ1638 mice underwent CLP and plasma was harvested and analyzed for IL-6 (FIG. 3A), IL-10 (FIG. 3B) and IL-12 (FIG. 3C) by ELISA. N=5/group. Data was analyzed by nonparametric T-Test. FIGS. 3D (plasma) and 3E (peritoneal lavage) demonstrate that NJ1638^{±} mice have no alteration in inflammatory cytokine production with pseudomonas peritonitis. No alterations in cytokines in IL-5 Tg or littermate controls. N=4-5/group.

FIG. 4 is a graph demonstrating the adoptive transfer of eosinophils improves survival in CLP. Littermate controls received either saline or 2×10⁵ eosinophils from sex matched NJ1638 mice 4 hours pre-CLP and monitored for survival.

FIG. 5 provides graphs demonstrating eosinophils and eosinophil granules kill P. aeruginosa ex vivo. P. aeruginosa was incubated with saline or eosinophils isolated from NJ1638^{±} mice at a multiplicity of infection (MOI) of 10 for 1 hour. Cells were lysed and plated for quantitative cultures (FIG. 5A). Protein extracts from eosinophil granules were incubated with P. aeruginosa at 1 mg/10⁶ colony forming units (CFU) (FIG. 5B). Data represents 3 independent experiments with 3 replicates per experiment. Data was analyzed by T-Test.

FIG. 6 provides a graph demonstrating IL-5 transgenic have improved survival with P. aeruginosa sepsis. NJ1638^{±} (n=15) or littermate controls (n=12) were injected with 10⁷ CFU P. aeruginosa intrapertioneally (ip) and monitored for survival.

FIGS. 7A and 7B provide a graph demonstrating IL-5 transgenic mice have improved bacterial clearance with P. aeruginosa. NJ1638^{±} or littermate controls were injected with 10⁷ CFU P. aeruginosa ip and blood and peritoneal lavage were harvested at 18 hours for quantitative cultures. For each experiment (3 mice/group) the average % reduction for NJ1638^{±} compared to controls was calculated. Data represent the mean for 3 independent experiments. FIG. 7C is a graph of circulating bacteria in wild-type mice which were adoptively transferred 2×10⁵ EOS or PBS and injected with PA ip and quantitative cultures taken from the peritoneal lavage (N=9/group). FIG. 7D is a graph of the peritoneal bacterial burden of PHIL^{±} mice or littermate controls which were injected with 107 CFU P. aeruginosa ip and monitored bacterial clearance (n=5/group). FIGS. 7E and 7F provide graphs which demonstrate that eosinophil granules kill P. aeruginosa in vitro and in vivo. FIG. 7E is a graph of P. aeruginosa CFU after protein extracts from eosinophil granules were incubated with P. aeruginosa at 1 mg/10⁶ CFU. FIG. 7F is a graph of the peritoneal bacterial burden of wild-type mice injected with P. aeruginosa ip and given eosinophil granule extract or vehicle control 1 hour after injection and quantitative cultures taken from peritoneal lavage (N=7/group). Data was analyzed by T-Test. FIGS. 7G (plasma) and 7H (peritoneal lavage) demonstrate that eosinophil granule extracts have no affect on inflammatory cytokine production in experimental pseudomonas peritonitis. N=4-5/group. FIG. 7I is a graph demonstrating that eosinophil granule extracts improve survival after CLP. Wild-type mice administered either vehicle or granule extract 2 hours post-CLP. N=10/group.

FIG. 8 is a graph demonstrating recombinant IL-5 increases myeloperoxidase (MPO). C57BL/6 mice (5 mice/group) were injected with 1 μg IL-5 ip or saline. Mice were harvested at 24 hours and peritoneal lavage MPO content assayed via ELISA. Data was analyzed via T-Test

FIGS. 9A and 9B are graphs demonstrating that recombinant IL-5 improves survival in CLP. In FIG. 9A, C57BL/6 mice were injected with 1 μg IL-5 ip or saline (solid) 4 hours pre (top solid) or 1 hour post (dashed line) CLP and monitored for survival for 14 days. In FIG. 9B, BC57BL/6 mice were injected with 1.5 μg IL-5 ip (squares, n=5) or saline (circles, n=7) 4 hours post 22 ga CLP and monitored for survival for 14 days. No additional deaths after 6 days. Data was analyzed by Kaplan Meier method.

FIGS. 10A and 10B provides graphs demonstrating that IL-5 stimulates PMN and monocyte development and improves survival in CLP independent of eosinophils. FIG. 10A provides graphs of a flow cytometry analysis of spleens from wild-type and NJ1638^{±}/PHIL^{±} mice at baseline. Data expressed as % PMN (LY6G) and % monocytes (% CD11B⁺). FIG. 10B depicts the percent survival of NJ1638^{±}/PHIL^{±} or littermate controls that underwent CLP. No deaths beyond 7 days. N=4-8/group. FIGS. 10C-10E demonstrate that endogenous IL-5 is protective in CLP. IL-5^{-/-} mice or age and sex matched littermate control underwent CLP for survival (FIG. 10C) or were harvested at 18 hours for quantitative culture (FIG. 10D) or cytokine analysis (FIG. 10E). N=10/group for survival and 4-5 for cytokines and cultures.

FIG. 11 provides graphs demonstrating murine neutrophils (PMNs) express IL-5Rα. C57BL/6 mice underwent CLP. Peritoneal lavage was harvested at 18 hours for flow cytometry. PMNs were identified by Ly6G and IL-5Rα via CD125. FIG. 11A is a representative dot plot and FIG. 11B is a representative histogram. The left peak represents isotype antibody staining.

FIG. 12A provides graphs demonstrating LPS upregulates macrophage expression of IL-5Rα. RAW 264.7 cells were incubated with vehicle (top row) or LPS (10 ng/ml) for the specified time points. Cells were harvested and stained with either isotype control (shaded), IL-5Rα (CD125; dark grey) or CTLA-4 (light grey). FIG. 12B provides a graph demonstrating IL-5Rα expression as part TRAF-6 dependent. RAW264.7 cells were stimulated with CpG (asterisk), PBS (C) or CpG+P13 (CP) and stained for IL-5Rα. Shaded grey area represents isotype control.

FIG. 13 demonstrates that IL-5 induces signaling in murine macrophages. Thioglycollate elicited peritoneal macrophages were primed with IFN-α (10 U/ml) for 24 hours and then stimulated with either vehicle or IL-5 for 24 hours. FIG. 13A is an image of an immunoblot of nuclear extracts for STAT-1. FIG. 13B is a graph of the analysis of IL-6 (left) and IL-12 (right) in culture supernatant via ELISA. FIGS. 13C and 13D demonstrate that IL-5 increases intracellular calcium in murine macrophages and PMNS. Lipopolysaccharide primed raw cells (FIG. 13D, left) or PMNS (FIG. 13D, right) were stimulated with fMLP (f-Met-Leu-Phe), IL-5, or PBS (buffer) and intracellular calcium expression was measured using fluorophore dye.

FIG. 14 is a graph demonstrating that IL-5 levels are increased in human sepsis. Plasma from healthy subjects (N=13) or subjects with sepsis (N=40) were assayed within 24 hours of intensive care unit (ICU) admission for IL-5 via ELISA. Data was analyzed via T-Test.

FIG. 15 provides graphs demonstrating increased IL-5 levels are associated with improved outcome in human sepsis. Plasma from subjects with sepsis (N=40) were assayed within 24 hours of ICU admission for IL-5 via ELISA. FIG. 15A presents the levels of IL-5 in subjects never intubated or intubated at any time during their ICU stay. FIG. 15B presents levels of IL-5 in survivors and non-survivors. Data was analyzed by 2-tailed T-Test.

FIGS. 16A-16C provide graphs demonstrating IL-5Rα expression on human cells. FIG. 16A shows THP1 cells stimulated with LPS for 48 hours and stained for IL-5Rα (unshaded trace). FIG. 16B shows the expression of IL-5Rα (unshaded trace) on CD14⁺ PMNs from a septic patient. FIG. 16C shows the expression of IL-5Rα on CD16⁺ monocytes from a septic patient. Grey represents isotype control.

### DETAILED DESCRIPTION OF THE INVENTION

Eosinophils (EOS) are a granulocyte subset which comprise 1-3% of circulating leukocytes in normal individuals. Eosinophils are derived from the same myeloid stem cell (CD34⁺) as PMNs and monocytes (Rothenberg et al. (2006) Annu. Rev. Immunol., 24:147-74). Interleukin-5 (IL-5), in combination with GM-CSF, is responsible for shifting myeloid precursors towards eosinophil development while GM-CSF and IL-3 will drive cells towards PMN development (Lopez et al. (1986) J. Exp. Med., 163:1085-99; Martinez-Moczygemba et al. (2003) J. Allergy Clin. Immunol., 112:653-66; Afshar et al. (2007) Curr. Opin. Pulm. Med., 13:414-21). Once matured, eosinophils express a variety of cell surface receptors, the most notable of which is CCR3 (Eotaxin receptor). This, combined with their unique granular staining and size characteristics, allows for reliable identification of eosinophils by flow cytometry (Rothenberg et al. (2006) Annu. Rev. Immunol., 24:147-74).

The eosinophil granule is comprised of a core of Major Basic Protein (MBP) surrounded by a matrix containing eosinophil peroxidase (EPO), eosinophil cationic protein (ECP) and eosinophil derived neurotoxin (EDN). In rodents, ECP and EDN are represented by a family of eosinophil associated ribonucleases (EARS) of which there are currently at least 11 members (Rothenberg et al. (2006) Annu. Rev. Immunol., 24:147-74). Furthermore, recent studies suggest eosinophils granules, similar to PMNs, contain the antibacterial peptide, BPI (Calafat et al. (1998) Blood 91:4770-5). The function of eosinophil granule proteins has been the subject of much study and debate. One important function of eosinophil granule proteins is for the host control of parasitic infections (Rothenberg et al. (2006) Annu. Rev. Immunol., 24:147-74; Specht et al. (2006) Infect. Immun., 74:5236-43). However, in contrast to this potential beneficial function, granule proteins can be toxic to a number of tissues, including heart, bronchial epithelium and vascular endothelium (Afshar et al. (2007) Curr. Opin. Pulm. Med., 13:414-21; Walsh, G. M. (2001) Curr. Opin. Hematol., 8:28-33; Wang et al. (2006) Blood 107:558-65).

Aside from release of their granule proteins, eosinophils have numerous other immune effector functions. Eosinophils are capable of producing and responding to numerous cytokines involved in all phases of the host immune response including IL-4, IL-13, TNF-α, IL-6, IL-10, TGF-β, MIP-1α and GM-CSF (Walsh, G. M. (2001) Curr. Opin. Hematol., 8:28-33; Velazquez et al. (2000) Immunology 101:419-25; Lacy et al. (2000) Chem. Immunol., 76:134-55). Eosinophils can also function as antigen presenting cells for T-cells and B-cells (Shi, H. Z. (2004) J. Leukoc. Biol., 76:520-7; Woerly et al. (1999) J. Exp. Med., 190:487-95). In addition, they express numerous cell surface molecules involved in cell-cell contact capable of further regulating the immune response, including VCAM and the costimulatory molecules CD80 and CD86 all of which have been described to have important immunomodulatory roles in a variety of disease states including sepsis (Woerly et al. (1999) J. Exp. Med., 190:487-95; Tamura et al. (1996) Scand. J. Immunol., 44:229-38; Chihara et al. (1999) J. Allergy Clin. Immunol., 103:S452-6; Schleimer et al. (1992) J. Immunol., 148:1086-92; Nolan et al. (2008) Am. J. Respir. Crit. Care Med., 177:301-8; Hoshino et al. (2002) J. Exp. Med., 195:495-505).

In vivo, eosinophils are traditionally believed to be central to the host response to parasitic infections, particularly nematodes. This stems from their presence at the site of infection and the anti-parasitic properties of their granule proteins. However, the ability of eosinophils to respond to Th2 cytokines and IgE stimulation of eosinophils has implicated eosinophils as a pathological effector cell in a variety of allergic diseases, most notably of which is asthma (Rothenberg et al. (2006) Annu. Rev. Immunol., 24:147-74). Interestingly, the role of viral infections in precipitating asthma exacerbations, and the presence of eosinophils in the lungs during these exacerbations, has led investigators to question whether eosinophils also play a role in the host's innate immune response to viral infections. This hypothesis has been supported by the presence of multiple TLRs, including TLR3 and TLR7 on eosinophils (Wong et al. (2007) Am. J. Respir. Cell. Mol. Biol., 37:85-96). Furthermore, stimulation of these receptors results in eosinophils activation and viral killing. This is mediated through the granule proteins ECP and EPO (Phipps et al. (2007) Blood 110:1578-86; Rosenberg et al. (2001) J. Leukoc. Biol., 70:691-8).

The expression of TLR2 and TLR4 on eosinophils also suggests a potentially important role for eosinophils in the host response to traditional bacterial infections as well. In vitro, human eosinophils are capable of recognizing gram⁺, gram⁻, mycobacteria and fungi with subsequent degranulation and killing (Ishihara et al. (2003) Biochim. Biophys. Acta., 1638:164-72; Lehrer et al. (1989) J. Immunol., 142:4428-34; Svensson et al. (2005) Microbes Infect., 7:720-8; Persson et al. (2001) Infect. Immun., 69:3591-6; Borelli et al. (2003) Infect. Immun., 71:605-13; Inoue et al. (2005) J. Immunol., 175:5439-47). Bacterial killing is mediated by superoxide production from EPO and eosinophil granule proteins. In addition, a direct antibacterial effect has been ascribed to ECP (EARS for rodents) and MBP (Ishihara et al. (2003) Biochim. Biophys. Acta., 1638:164-72; Lehrer et al. (1989) J. Immunol., 142:4428-34). Finally, the recent description of BPI in eosinophil granules makes this another potential candidate (Calafat et al. (1998) Blood 91:4770-5).

Aside from these in vitro observations, little is known about the role for eosinophils in the innate immune response to bacterial infections in vivo. Indirect evidence has yielded mixed results. In murine studies, induction of tissue eosinophilia via OVA sensitization impairs Pseudomonas clearance and killing (Beisswenger et al. (2006) J. Immunol., 177:1833-7). However, it is impossible to fully segregate the effects of eosinophils from those of TH2 cell activation and cytokine production. In humans, patients with primary hypereosinophilic syndromes rarely present with bacterial superinfections. Conversely, subjects with asthma have an increase in invasive pneumocccoal infections (Talbot et al. (2005) N. Engl. J. Med., 352:2082-90). However, it is difficult to interpret these data due to the high prevalence of glucocorticoid use for primary treatment for these disorders which can also impair other innate immune effector cells, including PMNs.

Interestingly, in mice and normal individuals, the presence of bacteremia is associated with a relative eosinopenia (Bass et al. (1980) J. Clin. Invest., 65:1265-71; Setterberg et al. (2004) Clin. Infect. Dis., 38:460-1; Weiner et al. (1952) Am. J. Med., 13:58-72; Bass, D. A. (1975) J. Clin. Invest., 56:870-9). Numerous case series describe eosinopenia with acute infections with the severity of infection and mortality correlating with the degree of eosinopenia (Weiner et al. (1952) Am. J. Med., 13:58-72). Interestingly, early reports also describe eosinophilia as a marker of resolution (Weiner et al. (1952) Am. J. Med., 13:58-72). These observations were confirmed in a more recent study where sepsis was associated with loss of circulating eosinophils and CCR3 (eotaxin receptor) expression (both on eosinophils and T-cells), with non-survivors having persistently lower levels compared to survivors (Venet et al. (2004) Clin. Immunol., 113:278-84). The mechanism of this eosinopenia is less clear. Early studies ascribe this to host mediators stimulated by bacterial infection (Bass et al. (1980) J. Clin. Invest., 65:1265-71). While the persistence of eosinopenia in adrenalectomized animals eliminates endogenous cortisol as the sole mediator of this effect, the specific compound was not identified (Bass et al. (1980) J. Clin. Invest., 65:1265-71; Bass, D. A. (1975) J. Clin. Invest., 55:1229-36). Consequently, the loss of eosinophils with severe bacterial infections was initially believed to be a marker of severity of infection. However, an equally plausible explanation is failure to maintain circulating eosinophils predisposes subjects to a worse outcome possibly due to impairment of host defense and thus failure to control bacterial replication. However, in the absence of studies directly addressing the role of eosinophils in the absence of the aforementioned confounders, it is difficult to draw any definitive conclusions.

Eosinopoiesis is in large part dependent upon IL-5 (Lopez et al. (1986) J. Exp. Med., 163:1085-99; Martinez-Moczygemba et al. (2003) J. Allergy Clin. Immunol., 112:653-66; Afshar et al. (2007) Curr. Opin. Pulm. Med., 13:414-21). This cytokine is part of a family of growth factors which includes IL-3 and GM-CSF (Martinez-Moczygemba et al. (2003) J. Allergy Clin. Immunol., 112:653-66). IL-5 is produced by numerous cell types including eosinophils, T-cells, mast cells, NK cells and macrophages (Martinez-Moczygemba et al. (2003) J. Allergy Clin. Immunol., 112:653-66). Its production is upregulated in part by the TH2 cytokines including IL-4 and IL-13 (Martinez-Moczygemba et al. (2003) J. Allergy Clin. Immunol., 112:653-66).

The IL-5 receptor (IL-5R) is a dimer. The α-subunit (IL-5Rα, CD125) is unique to IL-5 binding and signaling (Martinez-Moczygemba et al. (2003) J. Allergy Clin. Immunol., 112:653-66). The β-subunit (common β-chain, CD131), is shared by the IL-3, IL-5 and GM-CSF receptors and is responsible for much of the intracellular signaling (Martinez-Moczygemba et al. (2003) J. Allergy Clin. Immunol., 112:653-66). Ligation of IL-5R activates numerous intracellular signaling cascades. Most notably are phosphorylation of STAT-1, STAT-Sa/b, activation of p38 and ERK MAP kinase members as well as activation of P-I-3Kinase (Martinez-Moczygemba et al. (2003) J. Allergy Clin. Immunol., 112:653-66; Caldenhoven et al. (1999) Mol. Cell Biol. Res. Commun., 1:95-101; Stomski et al. (1999) Blood 94:1933-42; Adachi et al. (1998) Am. J. Physiol., 275:C623-33; Liva et al. (2001) Neurochem. Res., 26:629-37; Pazdrak et al. (1995) J. Immunol., 155:397-402). However, in spite of the shared β-subunit, IL-5 induces distinct signaling events compared to IL-3 and GM-CSF in the same cells making it impossible to extrapolate any biological effects of IL-5 stimulation from data obtained with IL-3 or GM-CSF.

The main cell types expressing IL-5Rα are eosinophils and eosinophil precursors. A major function for IL-5, in combination with IL-3 and GM-CSF, is to shift CD34⁺ myeloid progenitor cells towards eosinophil development (Rothenberg et al. (2006) Annu. Rev. Immunol., 24:147-74). This eosinopoetic role for IL-5 is confirmed by the hyper-eosinophilia observed in IL-5 overexpressing mice (NJ1638^{±}), and the severe attenuation in circulating eosinophils after allergen challenge in IL-5^{-/ -} mice (Lee et al. (1997) J. Immunol., 158:1332-44; Foster et al. (1996) J. Exp. Med., 183:195-201; Kopf et al. (1996) Immunity 4:15-24). IL-5 also acts as a chemoattractant and anti-apoptotic factor for eosinophils, recruiting and prolonging survival of eosinophils to sites of allergic inflammation (Stem et al. (1992) J. Immunol., 148:3543-9; Kagami et al. (2000) Blood 95:1370-7; Lilly et al. (1996) Am. J. Physiol., 270:L368-75). This was most evident in one study where nebulized IL-5 resulted in a nearly 6-fold increase in BAL eosinophils in asthmatics (Shi et al. (1998) Am. J. Respir. Crit. Care Med., 157:204-9). IL-5 is also required for host protection against numerous parasitic infections, and in most cases, this protection is due to altered eosinophil production and recruitment (Ramalingam et al. (2003) Exp. Parasitol., 105:131-9; Korenaga et al. (1991) Immunology 72:502-7). The other traditional cell type responsive to IL-5 stimulation is the B-cell. In particular, B-1 cells require IL-5 for IgA isotype class switching and subsequent generation of mucosal immunity (Moon et al. (2004) J. Immunol., 172:6020-9; Hiroi et al. (1999) J. Immunol., 162:821-8).

However, recent data suggest other cell types may be capable of responding to IL-5 stimulation providing a broader array of disorders potentially affected by IL-5 signaling. For example, IL-5 is required for generation of effective cytotoxic CD8⁺ cells in certain models (Apostolopoulos et al. (2000) Eur. J. Immunol., 30:1733-9). In addition, other studies document IL-5Rα gene expression in vascular smooth muscle, airway smooth muscle, astrocytes and airway epithelial cells (Wen et al. (2003) J. Allergy Clin. Immunol., 111:1307-18; Andrew et al. (2003) Environ. Health Perspect., 111:825-35; Colotta et al. (1993) Exp. Cell Res., 206:311-7; Awatsuji et al. (1993) J. Neurosci. Res., 34:539-45).

Of interest is the potential for IL-5Rα expression on other myeloid cells pivotal to the innate immune response in sepsis. Numerous studies suggest monocytes and macrophages are capable of responding to IL-5 stimulation. The mouse macrophage cell line, J774, demonstrates upregulation of multiple genes in response to IL-5 stimulation, including Platelet Derived Growth Factor a, Hypoxia Induced Gene 1 and various ribosomal proteins (Stumpo et al. (2003) Cytokine 24:46-56). Further, there was only partial overlap between IL-4, IL-5 and IFN-γ induced genes, showing a relative specificity for IL-5 stimulation (Stumpo et al. (2003) Cytokine 24:46-56). Functionally, IL-5 induces proliferation and inhibits apoptosis in the mouse macrophage cell line, RAW 264.7 and primary murine microglial cells. These effects were similar to those observed with GM-CSF stimulation, and were completely blocked by a monoclonal antibody to IL-5 (Ringheim, G. E. (1995) Neurosci. Lett., 201:131-4). These results were confirmed by another group where IL-5 induced cellular proliferation and nitric oxide (NO) production from primary rat microglial cells (Liva et al. (2001) Neurochem. Res., 26:629-37). However, no data exists as to the significance of IL-5 stimulation of mononuclear cells in vivo.

Neutrophils (PMNs) are another innate immune effector cell which may be capable of responding to IL-5 stimulation. IL-5Rα mRNA has been found in human PMNs (Suttmann et al. (2003) Infect. Immun., 71:4647-56). In addition, horses with heaves, a disorder similar to COPD, express high levels of IL-5Rα on circulating PMNs (Dewachi et al. (2006) Vet. Immunol. Immunopathol., 109:31-6). In a murine model of filariasis, IL-5^{-/-} mice, or blockade of IL-5 with a monoclonal antibody, impaired eosinophils and PMN recruitment to the site of infection (Al-Qaoud et al. (2000) Int. Immunol., 12:899-908). Further, tracheal administration of recombinant IL-5 to guinea pigs resulted in significant PMN recruitment into the alveolar space (Lilly et al. (1996) Am. J. Physiol., 270:L368-75). Finally, IL-5 has been shown to act as a chemoattractant for human PMNs in cell culture (Bober et al. (1995) Clin. Exp. Immunol., 99:129-36).

Hereinbelow, an IL-5 transgenic mouse was utilized to test the role for eosinophils and IL-5 in polymicrobial sepsis. In vivo data is provided which demonstrates a functional and protective role for eosinophils and IL-5 in the innate immune response to polymicrobial sepsis with the latter being mediated in part independently of the presence of EOS. The data also provide important information regarding the in vivo role for eosinophils and their maturation factor, IL-5, in the innate immune response to polymicrobial sepsis. Initial studies focused on the NJ1638^{±} (IL-5 transgenic) mouse which had improved survival and bacterial clearance in CLP. This indicated an important and novel role for eosinophils and/or IL-5 in the host response to polymicrobial sepsis.

To ascertain the precise role for eosinophils, a method of flow sorted eosinophil isolation from whole blood from NJ1638^{±} mice was developed for ex vivo and adoptive transfer studies. While prior studies suggest an in vitro bactericidal role for eosinophils (Svensson et al. (2005) Microbes Infect., 7:720-8; Persson et al. (2001) Infect. Immun., 69:3591-6), an in vivo antibacterial function and anti-pseudomonal activity for eosinophils is shown hereinbelow. This effect was mediated in part by the eosinophil granule proteins (Ishihara et al. (2003) Biochim. Biophys. Acta., 1638:164-72; Lehrer et al. (1989) J. Immunol., 142:4428-34). These findings were extended to document an in vivo antibacterial function for eosinophils, evidenced by the improved clearance and survival of Pseudomonas aeruginosa infected NJ1638^{±} mice as well as improved clearance of Pseudomonas aeruginosa in WT mice after adoptive transfer of eosinophils. Finally, this was confirmed in the more physiologically relevant model of sepsis, CLP, with improved survival in littermate control mice after adoptive transfer of eosinophils from sex matched NJ1638^{±} siblings. Together, these data demonstrate an effective in vivo antibacterial role for eosinophils in the host response to bacterial infections.

The potent in vivo role for eosinophils, however, could not fully exclude an equally important role for IL-5 in the NJ1638^{±} mice as well. Surprisingly, PMNs and monocytes/macrophages express high levels of the IL-5Rα after CLP as documented by flow cytometry and immunoblot. This was modeled in vitro with LPS stimulation of murine macrophages. The receptor is biologically active as evidence by IL-6 production and STAT-1 nuclear translocation, a known downstream effect of IL-5 stimulation in other cell types (Pazdrak et al. (1995) J. Immunol., 155:397-402). In vivo, IL-5 was capable of rescuing mice when administered pre or post-CLP suggesting a potentially novel therapeutic role for IL-5 pharmacological administration. Interestingly, this was not due to increased eosinophil recruitment. Rather, the increase in PMN recruitment observed with IL-5 administration coupled with the presence of a biologically active IL-5Rα on PMNs and monocytes, indicates IL-5 is an immunomodulator capable of affecting multiple cell types.

Important information as to the role of IL-5 in human sepsis is also provided hereinbelow. The finding of higher levels of IL-5 in survivors and non-intubated septic patients (compared to non-survivors and intubated subjects respectively), is in agreement the finding of higher levels of IL-5 in mice subjected to sublethal CLP compared to lethal CLP. This indicates lethal sepsis is associated with either a loss and/or inadequate upregulation of IL-5 production and provides further support for the administration of IL-5 as a therapeutic intervention in human sepsis. Furthermore, in another embodiment, IL-5 levels in a patient (e.g., from the blood) may be measured as a prognostic indicator for sepsis, wherein a decreased IL-5 level (e.g., the IL-5 levels are similar to IL-5 sepsis non-survivors) is indicative of a poor prognosis and increased mortality and wherein increased levels of IL-5 (e.g., the IL-5 levels are similar to IL-5 sepsis survivors) is indicative a good prognosis and decreased mortality.

### I. Definitions

The term “sepsis” as used herein refers to the systemic inflammatory response to infection. In other words, “sepsis” may refer to a systemic inflammatory response plus a documented infection (e.g., a subsequent laboratory confirmation of a clinically significant infection such as a positive culture for an organism) (see, e.g., American College of Chest Physicians Society of Critical Care Medicine (1997) Chest 101:1644-1655). The “systemic inflammatory response” is the body's overwhelming response to a noxious stimulus. The current definition is characterized by the following non-specific changes in the adult human body: 1) fast heart rate (tachycardia, heart rate >90 beats per minute), 2) low blood pressure (systolic <90 mmHg or MAP <65 mmHg), 3) low or high body temperature (<36 or >38° C.), 4) high respiratory rate (>20 breaths per minute), and 5) low or high white blood cell count (<4 or >12 billion cells/liter). When an identified infectious pathogen causes the inflammatory response, the resultant inflamed state is referred to as sepsis. Infectious agents which can cause sepsis include bacteria, viruses, fungi, and parasites.

As used herein, the term “sepsis” includes all stages of sepsis including, but not limited to, the onset of sepsis, severe sepsis, septic shock and multiple organ dysfunction associated with the end stages of sepsis. The “onset of sepsis” refers to an early stage of sepsis (e.g., prior to a stage when conventional clinical manifestations are sufficient to support a clinical suspicion of sepsis). “Severe sepsis” refers to sepsis associated with organ dysfunction (Bota et al. (2002) Intens. Care Med. 28:1619-1624; Ferreira et al. (2005) J. Amer. Med. Assoc. 286:1754-1758), hypoperfusion abnormalities, or sepsis-induced hypotension. Hypoperfusion abnormalities include, but are not limited to, lactic acidosis, oliguria, or an acute alteration in mental status. “Septic shock” refers to sepsis-induced hypotension that is not responsive to adequate intravenous fluid challenge and with manifestations of peripheral hypoperfusion. “Septic shock” may also be defined as severe sepsis accompanied by acute circulatory failure characterized by persistent arterial hypotension (a systolic arterial pressure below 90 mm Hg).

The term “treat” as used herein refers to any type of treatment that imparts a benefit to a patient afflicted with a disease, including improvement in the condition of the patient (e.g., in one or more symptoms), delay in the progression of the condition, etc.

The phrase “effective amount” refers to that amount of therapeutic agent that results in an improvement in the patient's condition.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

### II. Administration

In accordance with the instant invention, IL-5 is administered to a patient to treat sepsis and/or inhibit the onset or progression of sepsis. In a preferred embodiment, IL-5 is administered as a protein. However, the instant invention also encompasses the administration of IL-5 encoding nucleic acid (e.g., gene therapy) or cells expressing IL-5. As used herein, the term “IL-5” will refer to the protein IL-5. In a particular embodiment, IL-5 is administered to the patient at a concentration and frequency sufficient to recruit monocytes and/or PMNs (neutrophils), but, optionally, insufficient to recruit eosinophils. In another embodiment, the IL-5 is administered to increase blood (plasma) levels to at least about 3 μg/ml, at least about 4 μg/ml, at least about 5 μg/ml, or more.

In another embodiment of the instant invention, IL-5 is administered to a patient to recruit monocytes and/or PMNs (neutrophils) (increase the number of these cells at the site of administration as compared to levels prior to administration). The administration of IL-5 to recruit monocytes and/or PMNs can be used to treat an infection (e.g., a bacterial infection).

IL-5 may be prepared in a variety of ways, according to known methods. For example, the protein may be purified from appropriate sources, e.g., transformed bacteria, cultured animal cells or tissues, or animals (e.g., by immunoaffinity purification methods). The availability of nucleic acid molecules encoding IL-5 enables production of the protein using in vitro expression methods and cell-free expression systems known in the art. In vitro transcription and translation systems are commercially available, e.g., from Promega Biotech (Madison, Wis.) or Gibco-BRL (Gaithersburg, Md.).

Alternatively, larger quantities of IL-5 may be produced by expression in a suitable prokaryotic or eukaryotic system. For example, a DNA molecule encoding for IL-5 may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

IL-5 produced by gene expression in a recombinant prokaryotic or eukaryotic system may be purified according to methods known in the art. A commercially available expression/secretion system can be used, whereby the recombinant protein is expressed and secreted from the host cell, and readily purified from the surrounding medium by any method known in the art. For example, the recombinant protein may be purified by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

IL-5, prepared by the aforementioned methods, may be analyzed according to standard procedures. For example, such protein may be subjected to amino acid sequence analysis, according to known methods.

Exemplary amino acid sequences of human IL-5 are known (see, e.g., GenBank Accession Nos. AAA98620, AAK19759, AAA74469, and P05113). An IL-5 amino acid sequence may have 75%, 80%, 85%, 90%, 95%, 97%, or 99% homology with any of these sequences.

IL-5 will generally be administered to a patient (i.e., human or animal subject) in a composition with a pharmaceutically acceptable carrier. For example, IL-5 may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of IL-5 in the chosen medium may be varied and the medium may be chosen based on the desired route of administration of the pharmaceutical preparation. Except insofar as any conventional media or agent is incompatible with IL-5, its use in the pharmaceutical preparation is contemplated.

In yet another embodiment, the pharmaceutical compositions of the present invention can be delivered in a controlled release system, such as using an intravenous infusion, an implantable osmotic pump (e.g., a subcutaneous pump), a transdermal patch, liposomes, or other modes of administration. In a particular embodiment, a pump may be used (see Langer (Science (1990) 249:1527-1533); Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: N.Y. (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the desired site. Other controlled release systems are discussed in the review by Langer (Science (1990) 249:1527-1533).

The dose and dosage regimen of IL-5 that are suitable for administration to a particular patient may be determined by a physician considering the patient's age, sex, weight, general medical condition, and the specific condition for which IL-5 is being administered and the severity thereof. The physician may also take into account the route of administration, the pharmaceutical carrier, and the biological activity of the administered IL-5.

Selection of a suitable pharmaceutical preparation will also depend upon the mode of administration chosen. For example, IL-5 may be administered by direct injection into an area proximal to the infection. In this instance, a pharmaceutical preparation comprises the IL-5 dispersed in a medium that is compatible with the site of injection. IL-5 may be administered by any method such as intravenous injection into the blood stream, oral administration, or by subcutaneous, intramuscular or intraperitoneal injection. Pharmaceutical preparations for injection are known in the art. If injection is selected as a method for administering IL-5, steps must be taken to ensure that sufficient amounts of the molecules reach their target cells to exert a biological effect.

Pharmaceutical compositions containing IL-5 as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of IL-5 may be determined by evaluating the toxicity of the molecules in animal models. Various concentrations of IL-5 in pharmaceutical preparations may be administered to mice, and the minimal and maximal dosages may be determined based on the beneficial results and side effects observed as a result of the treatment. Appropriate dosage unit may also be determined by assessing the efficacy of IL-5 treatment in combination with other standard drugs. The dosage units of IL-5 may be determined individually or in combination with each treatment according to the effect detected.

The pharmaceutical preparation comprising IL-5 may be administered at appropriate intervals, for example, at least twice a day or more until the pathological symptoms are reduced or alleviated, after which the dosage may be reduced to a maintenance level. The appropriate interval in a particular case would normally depend on the condition of the patient.

Pharmaceutical compositions comprising IL-5 may be administered in coordination with at least one other agents used to treat sepsis. Other agents used to treat sepsis include, without limitation, anti-thrombin III, activated protein C (e.g., XIGRIS® (drotrecogin alfa); particularly for severe sepsis), BPI, anti-infectives, antibiotics (including, without limitation, β-lactams (e.g., penicillins and cephalosporins), vancomycins, bacitracins, macrolides (e.g., erythromycins), lincosamides (e.g., clindomycin), chloramphenicols, tetracyclines (e.g., immunocycline, chlortetracycline, oxytetracycline, demeclocycline, methacycline, doxycycline and minocycline), aminoglycosides (e.g., gentamicins, amikacins, and neomycins), amphotericins, cefazolins, clindamycins, mupirocins, sulfonamides and trimethoprim, rifampicins, metronidazoles, quinolones, novobiocins, polymixins, gramicidins, vancomycin, imipenem, meropenem, cefoperazone, cefepime, penicillin, nafcillin, linezolid, aztreonam, piperacillin, tazobactam, ampicillin, sulbactam, clindamycin, metronidazole, levofloxacin, a carbapenem, linezolid, rifampin, and metronidazole), and agents which alleviate/treat the symptoms associated with sepsis (supportive care; e.g., cardiovascular support, respiratory support, renal replacement therapy, glucose control, and analgesia (see, e.g., www.sepsis.com). IL-5 and the other anti-sepsis agents may be administered together in a single composition or may be administered in separate compositions. Additionally, IL-5 and the other anti-sepsis agents may be administered at the same time or on different schedules.

While compositions comprising IL-5 are exemplified hereinabove, compositions comprising eosinophil granules and/or extracts of eosinophil granules may used. For example, in a particular embodiment, compositions comprising eosinophil granules and/or extracts of eosinophil granules can be administered to a patient to treat sepsis and/or inhibit the onset or progression of sepsis The following examples are provided to illustrate certain embodiments of the invention. They are not intended to limit the invention in any way.

### EXAMPLE 1

### Hypereosinophilic Mice are Protected from the Lethality of Polymicrobial Sepsis

In order to determine the role for eosinophils in polymicrobial sepsis, IL-5 transgenic mice (NJ1638^{±}) were utilized. These mice, bred onto a C57BL/6 background, constitutively express IL-5 driven by the CD3δ promoter (Lee et al. (1997) J. Immunol., 158:1332-44). They have a profound peripheral eosinophilia compared to littermate controls by 6 wks of age (50% vs. 0.1%), which progresses throughout the lifespan of the mouse. However, the increase in eosinophils is not associated with in an increase in spontaneous eosinophils degranulation or activation, providing a model to study the function of nascent EOS (Lee et al. (1997) J. Immunol., 158:1332-44).

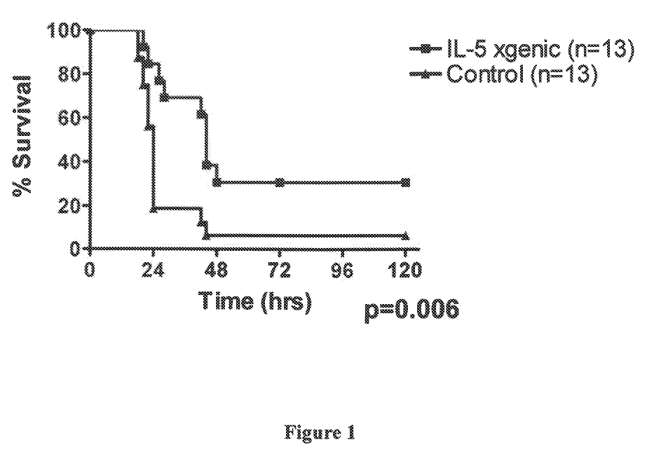

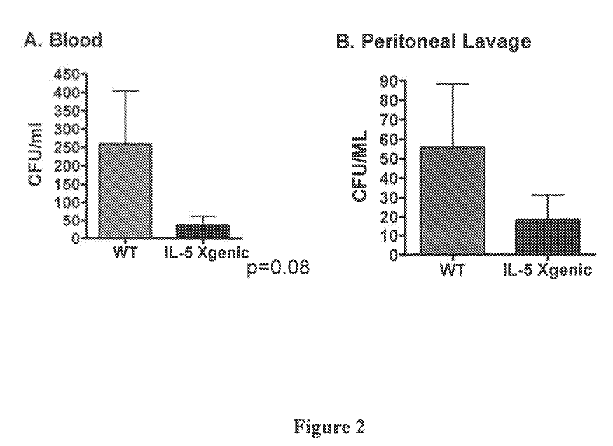

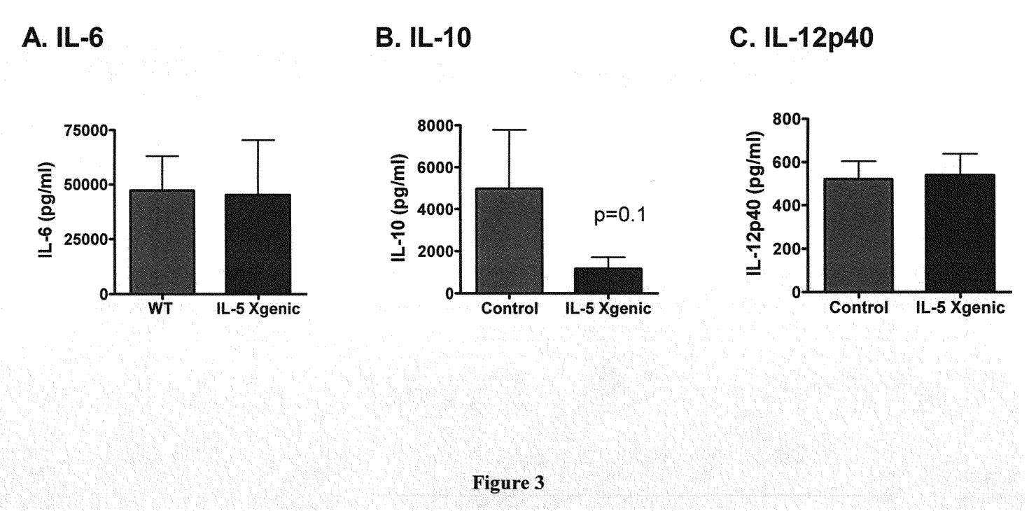

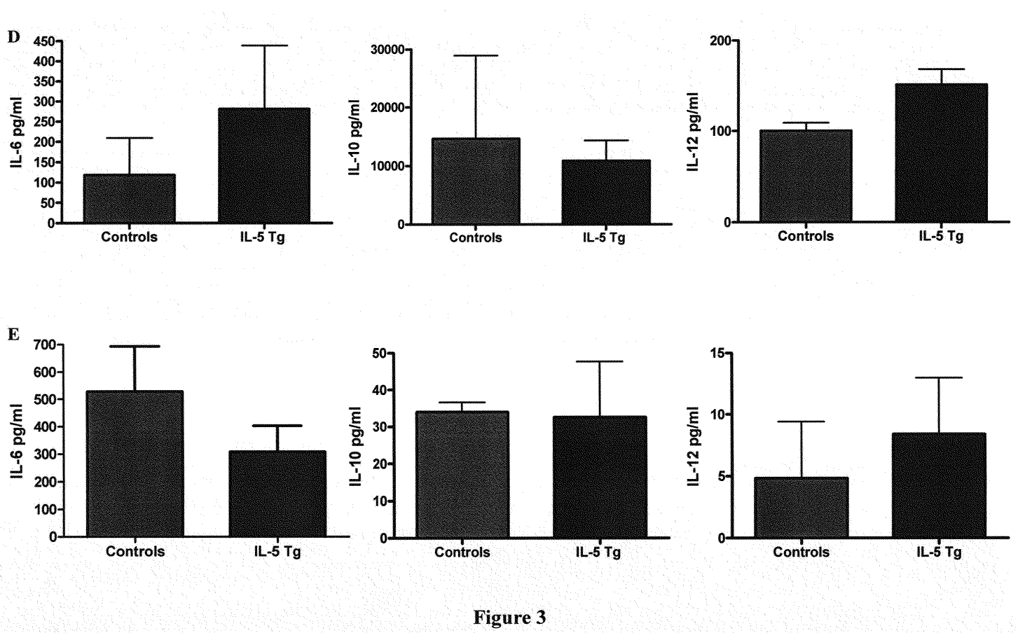

6-8 week male NJ1638^{±} or littermate controls underwent cecal ligation and puncture (CLP), a well accepted model of sepsis, with an 18 gauge needle. NJ1638^{±} mice showed a significant improvement in median (44 vs. 24 hours) and overall (30 vs. 7%) survival compared to littermate controls (p=0.006) (FIG. 1). Improvement in survival was associated with increased bacterial clearance. NJ1638^{±} mice demonstrated a reduction in peritoneal and blood bacterial burden compared to littermate controls 18 hours after CLP (FIG. 2). Interestingly, this was associated with little to no change in inflammatory cytokine production. NJ1638^{±} mice exhibited only a trend towards a reduction in plasma IL-10 and no difference in plasma or BALF IL-6 or IL-12 compared to littermate controls 18 hours after CLP (FIGS. 3A-3C). NJ1638^{±} mice also have no alteration in inflammatory cytokine production with pseudomonas peritonitis. No alterations in cytokines in IL-5 Tg or littermate controls were observed (FIGS. 3D and 3E).

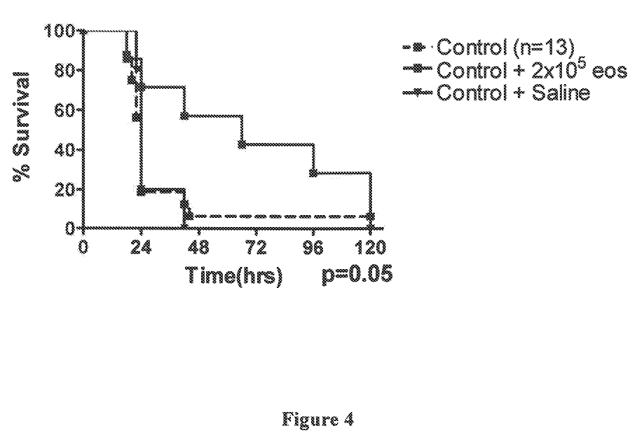

To better define the role of eosinophils, eosinophils were isolated from peripheral blood of 5 month old NJ1638^{±} mice, using percoll gradient centrifugation coupled with flow sorting based on FSC/SSC characteristics. This yields a population of approximately 97% eosinophils as determined by diffquik staining and flow cytometry (CCR3⁺). 2×10⁵ eosinophils or an equivalent volume of PBS intrapertioneally (ip) were adoptively transferred into sex-matched littermate controls 4 hours prior to CLP. Mice receiving eosinophils had a significant improvement in survival compared to saline treated controls, with survival nearly identical to NJ1638^{±} mice (FIG. 4). Together, these data indicate an important role for eosinophils in the innate immune response to polymicrobial sepsis, possibly through a direct antibacterial effect.

### EXAMPLE 2

### Eosinophils Have Antibacterial Properties in Vitro and in Vivo

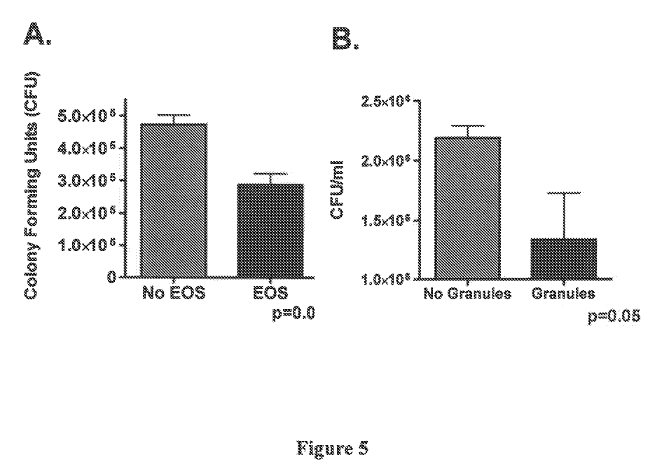

The potential antibacterial role for eosinophils was then studied, both in cell culture and using a well defined in vivo bacterial challenge. While a few reports document the presence of innate immune receptors (TLR 2, 4, 7) on human eosinophils, little is known about the antibacterial effects for eosinophils in vitro and none has investigated this in vivo (Wong et al. (2007) Am. J. Respir. Cell Mol. Biol., 37:85-96; Svensson et al. (2005) Microbes Infect., 7:720-8; Persson et al. (2001) Infect. Immun., 69:3591-6). Isolated eosinophils from NJ1638^{±} mice were incubated with either E. coli or Pseudomonas aeruginosa (FIG. 5) at an MOI of 10 for 1 hour. Eosinophils (compared to saline) reduced the number of viable bacteria by 50% at 1 hour (FIG. 5A). This was comparable to what was observed with isolated murine PMNs. Finally, this effect was in part due to the antibacterial activities of eosinophils derived granule proteins. Granules were isolated from murine eosinophils and sonciated to release the granule proteins. Similar to studies with human eosinophils, murine eosinophils granule proteins (1 mg/10⁶ Pseudomonas aeruginosa) resulted in an average 40% killing compared to vehicle treated controls (FIG. 5B).

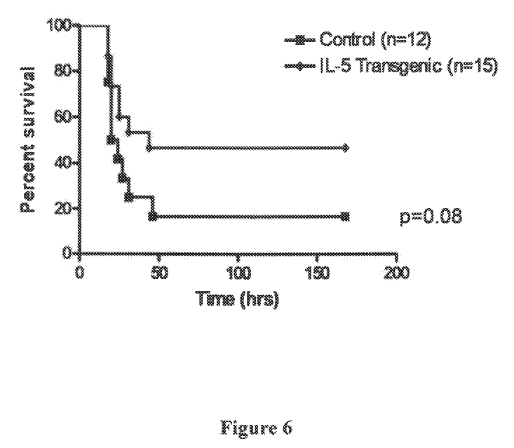

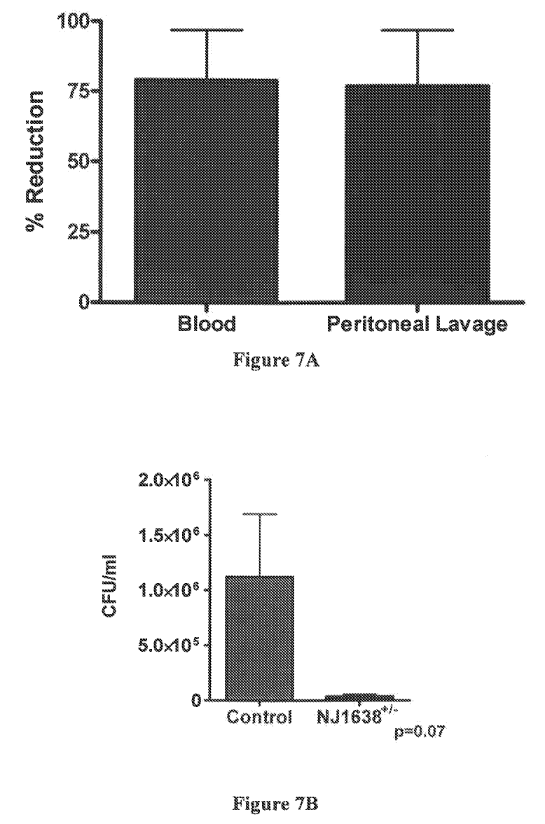

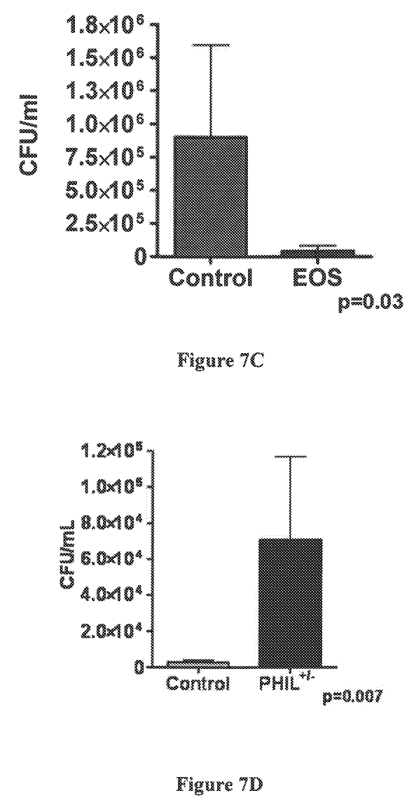

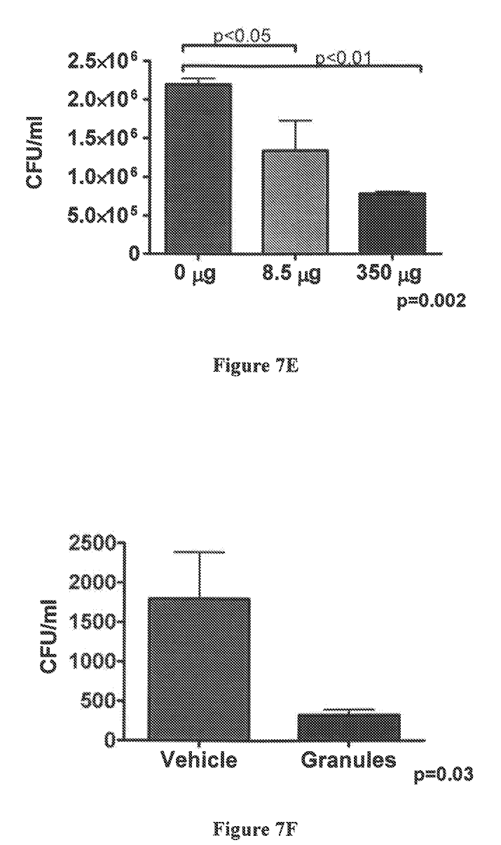

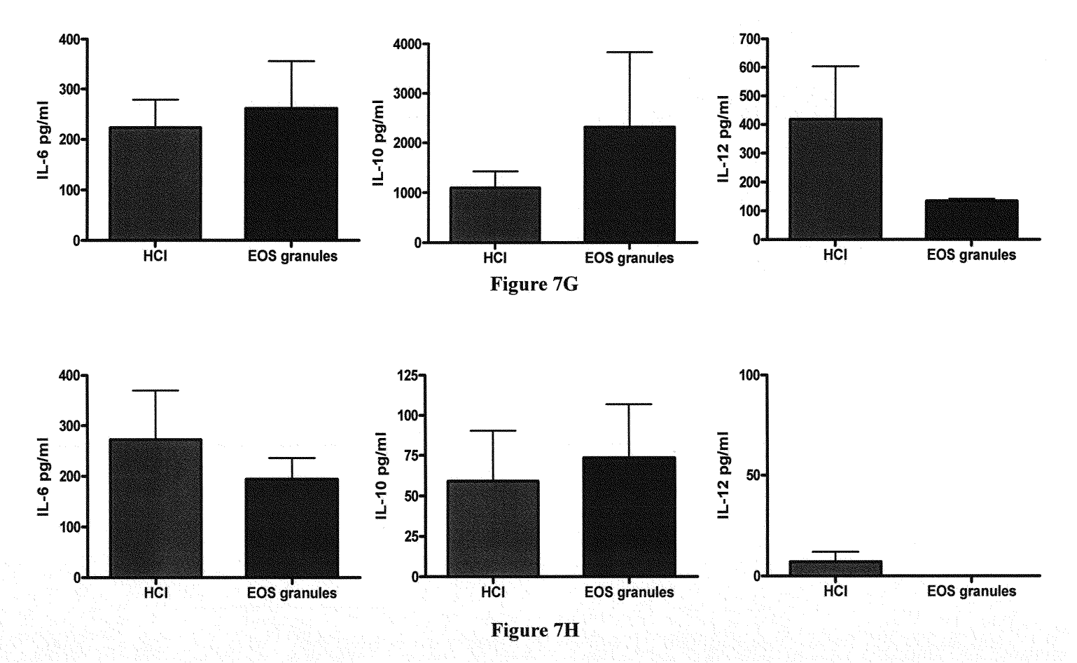

It was then determined whether this effect was relevant in vivo. NJ1638^{±} mice or littermate controls were injected with 10⁷ Pseudomonas aeruginosa intraperitoneally (ip) and monitored for survival and bacterial clearance. Similar to the results with the CLP model, NJ1638^{±} mice demonstrated an improvement in median and overall survival compared to littermate controls (FIG. 6). This improvement in survival was associated with an improvement in bacterial clearance with little change in local or systemic inflammatory cytokine production. NJ1638^{±} mice had a nearly 75% reduction in peritoneal and circulating bacterial burden compared to littermate controls as determined by quantitative cultures (FIGS. 7A and 7B). This effect was specific to eosinophils as adoptive transfer of eosinophils into C57BL/6 mice infected with Pseudomonas aeruginosa ip, resulted in a 2-log reduction in circulating bacteria compared to saline treated controls (FIG. 7C). Finally, PHIL^{±} mice, which lack endogenous EOS due to a dipetheria toxin transgene expressed under the EPO promoter, had a 20 fold increase in peritoneal bacterial burden compared to littermate controls (FIG. 7D). This was associated with an increase in detectable circulating bacteria in the blood (60% vs. 0%; p<0.04).

It was then determined whether similar antibacterial results could be obtained from eosinophil granules themselves. Granules were isolated from murine eosinophils and sonciated to release the granule proteins. Similar to studies with human eosinophils, murine eosinophil granule proteins (1 mg/10⁶ Pseudomonas aeruginosa) resulted in an average 40% killing in a dose dependent manner compared to vehicle treated controls (FIG. 7E). Granule proteins exerted a similar anti-bacterial affect as whole eosinophils in vivo as well. Administration of eosinophil granule proteins, 1 hour after administration of 10⁷ Pseudomonas aeruginosa ip, resulted in significant improvement in bacterial clearance compared to vehicle treated controls (FIG. 7F). Again, this had no affect on either local or systemic inflammatory cytokines (FIGS. 7G and 7H). Finally, administration of EOS granules 2 hours after CLP, while not changing overall mortality, did significantly increase median survival (p=0.03) (FIG. 7I).

Together, these data strongly indicate an antibacterial effect for eosinophils in vitro and in vivo. Further, these antibacterial properties are contained within eosinophil granules which can be administered in vivo as an antimicrobial therapy or as an adjuvant with another antimicrobial therapy.

### EXAMPLE 3

### Role of IL-5 in the Innate Immune Response to Polymicrobial Sepsis

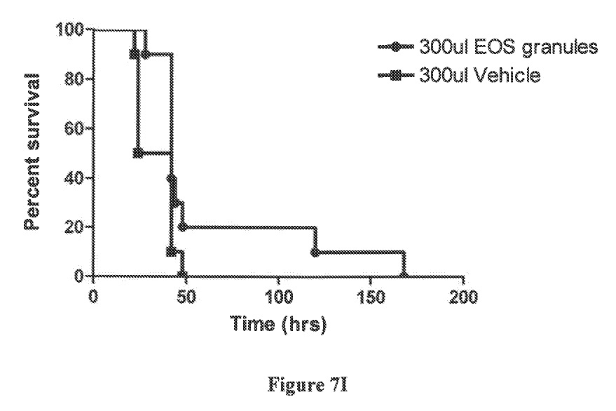

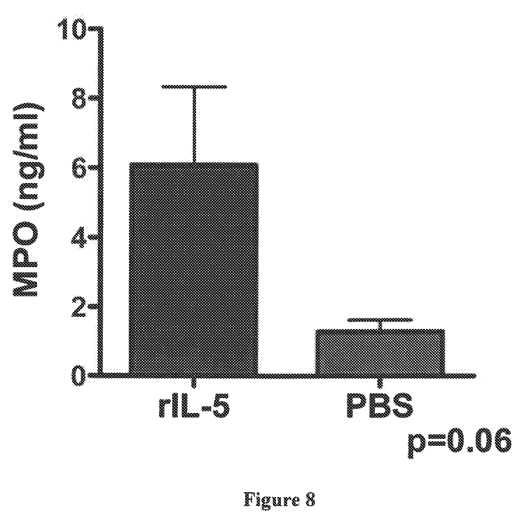

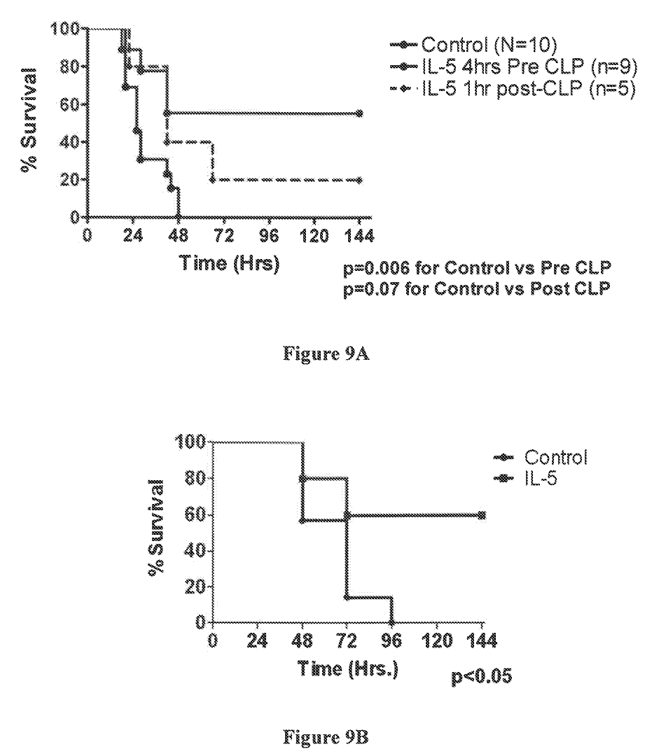

It was then determined whether exogenous administration of IL-5 would result in eosinophil recruitment similar to what is observed in NJ1638^{±} mice and thus further augment the host response in CLP. The levels of IL-5 in WT mice was first assessed at baseline and then after CLP. As expected, plasma levels of IL-5 were <10 pg/ml and undetectable in peritoneal lavage (PL) from unoperated mice. 18 hours after lethal CLP (19 gauge needle—90% mortality at 72 hours), there was no significant change in plasma or PL IL-5 levels compared to unoperated controls. In contrast, mice subjected to sublethal CLP (27 gauge needle—20% morality at 7 days) had higher levels of IL-5 in PL compared to mice subjected to lethal CLP (3.2 pg/ml vs. 0.0 pg/ml; p<0.0001). However, administration of IL-5 (1 μg) ip into unoperated WT mice failed to significantly increase the percent or absolute number of peritoneal or circulating eosinophils 4 and 24 hours after administration. Of note, this dose of IL-5 yielded plasma levels of approximately 100 pg/ml, which was significantly higher than saline treated controls (<10 pg/ml) and similar to what is observed in NJ1638^{±} mice (200 pg/ml), indicating that chronically elevated levels of IL-5 are required for effective eosinopoesis. Interestingly, IL-5 administration had a significant PMN chemotactic effect. Mice administered IL-5 ip had a significant increase in PMN recruitment to the peritoneal cavity by flow cytometry and as evidenced by an increase in PL myeloperoxidase (MPO) levels compared to saline treated controls (FIG. 8). Despite a lack of significant eosinophil recruitment (indeed, eosinophil recruitment likely requires chronically elevated levels of IL-5), administration of recombinant IL-5 (1 μg) 4 hours prior to CLP significantly improved median and overall (55.5% vs. 0%) survival in C57BL/6 mice compared to saline treated controls (p<0.001) (FIG. 9A). This effect was also observed with post-CLP treatment (1 hour) (FIG. 9A). In order to improve upon the post-CLP survival benefit, the experiments were repeated with a higher dose of IL-5 (1.5 μg) and a small gauge needle puncture (22 gauge), the latter allowing for a further delay in mortality to test for further delays in IL-5 administration. Mice receiving IL-5 4 hours post CLP had a significantly improved survival compared to vehicle treated controls (FIG. 9B). Together, these data indicate a therapeutic role for IL-5 administration as a rescue therapy for severe sepsis.

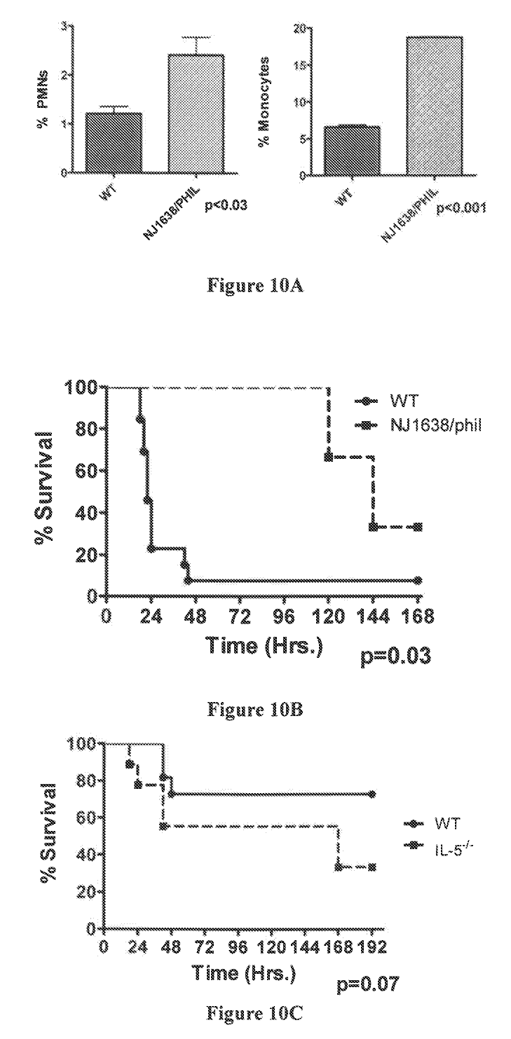

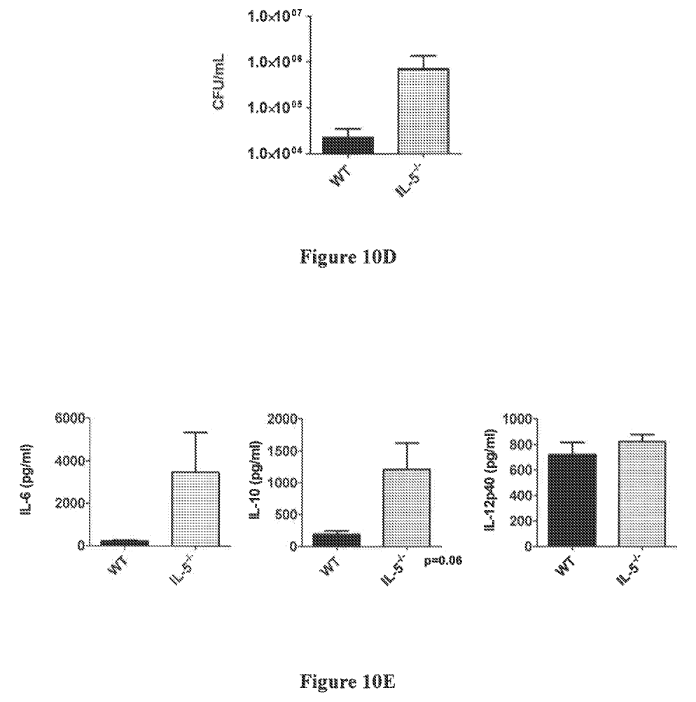

NJ1638^{±}/PHIL^{±} mice overexpress IL-5, but are incapable of producing mature eosinophils due to a diptheria-toxin transgene driven by the EPO promoter (Apostolopoulos et al. (2000) Eur. J. Immunol., 30:1733-9). Interestingly, these mice have expansion of PMNs and monocytes in the spleen and peripheral blood (FIG. 10A), thereby indicating IL-5 is capable of regulating non-eosinophil granulocyte lineages. These mice have improved survival compared to littermate controls after CLP (FIG. 10B). Finally, IL-5−/− mice had increased mortality compared to controls, further demonstrating a protective effect for IL-5 in sepsis (FIG. 10C). This increase in mortality was associated with trends towards a decrease in bacterial clearance (FIG. 10D) and increased inflammatory cytokine production (FIG. 10E).

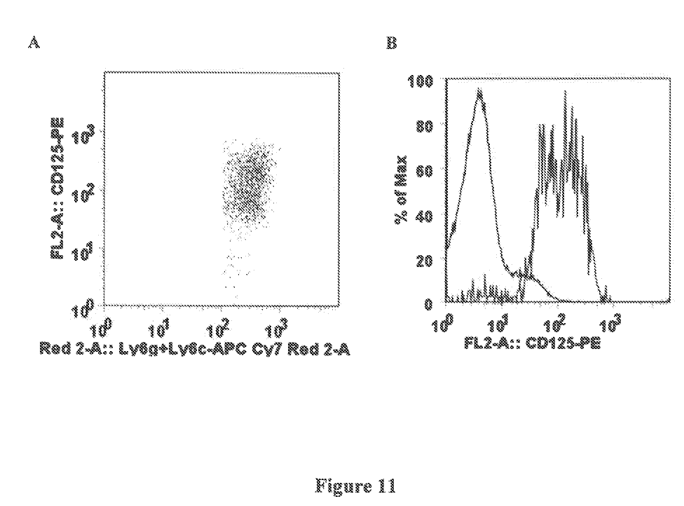

The ability of IL-5 to rescue mice in CLP and increase PMN recruitment in the absence of eosinophils, indicates other innate immune effector cells are responsive to IL-5 stimulation and thus express IL-5Rα. WT C57BL/6 mice had little expression of IL-5Rα on resident peritoneal macrophages (F4/80⁺) or circulating PMNs. However, 18 hours after CLP, peritoneal PMNs (Ly6G⁺) (FIG. 11) expressed high levels of IL-5Rα, with similar results seen with peritoneal macrophages (F4/80⁺) and circulating PMNs. There were no eosinophils observed in PL or blood on direct visualization and all of these cells were all CCR3⁻ eliminating the possibility these were contaminating eosinophils.

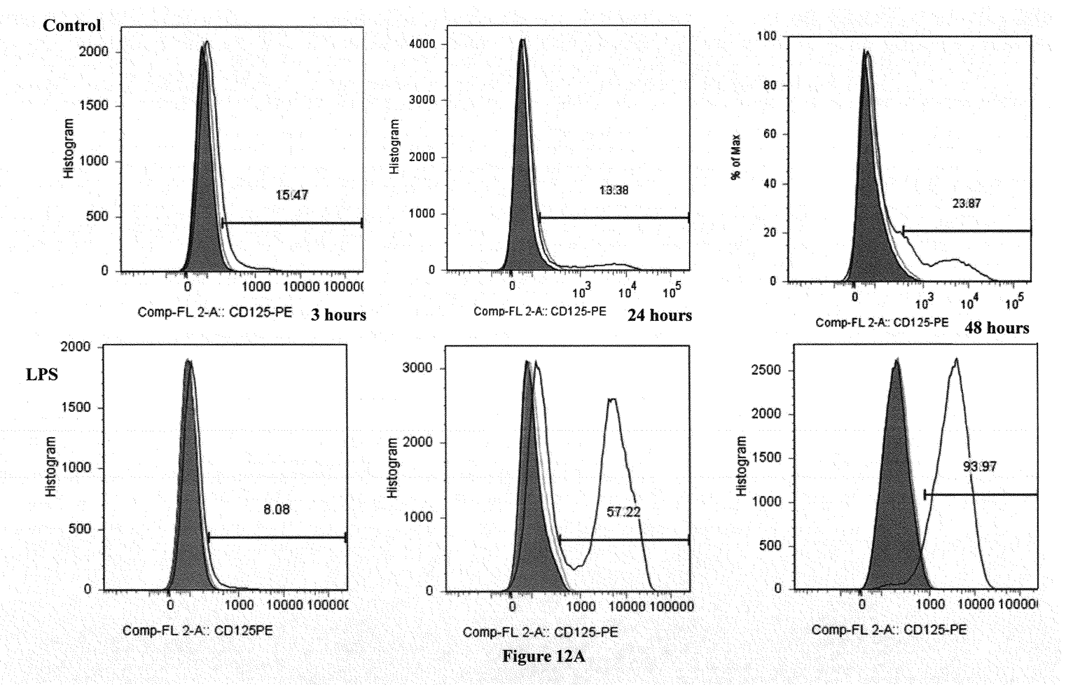

The regulation and functionality of the IL-5R on macrophages and PMNs was subsequently determined. Initial experiments utilized thioglycollate elicited peritoneal macrophages and the murine macrophage cell line, RAW 264.7. Neither cell type expressed IL-5Rα at baseline. However, IL-5Rα was significantly upregulated 48 hours after IFN-α (10 U/ml) or LPS (10 ng/ml; FIG. 12A) or CpG (FIG. 12B) stimulation. Flow cytometry results were confirmed via immunoblot of whole cell lysates. The ability of both TLR4 and TLR9 agonists to regulate IL-5Rα, suggests a prominent role for NF-kB. Treatment of cells with P13, a peptide derivative of vaccine A52R, which inhibits TRAF6 and thus NF-kB signaling, completely abolished CpG induced IL-5Ra upregulation (FIG. 12B).

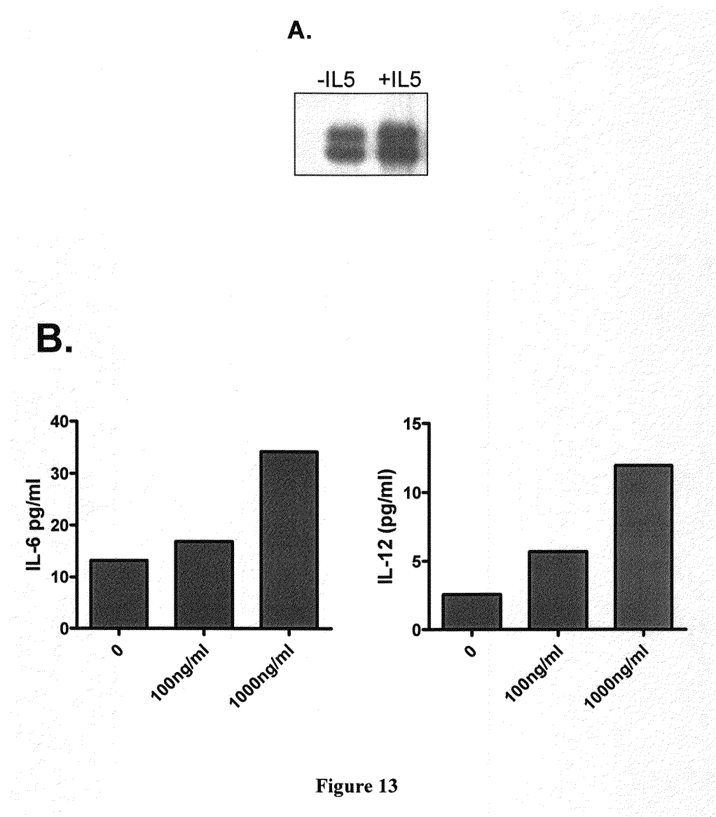

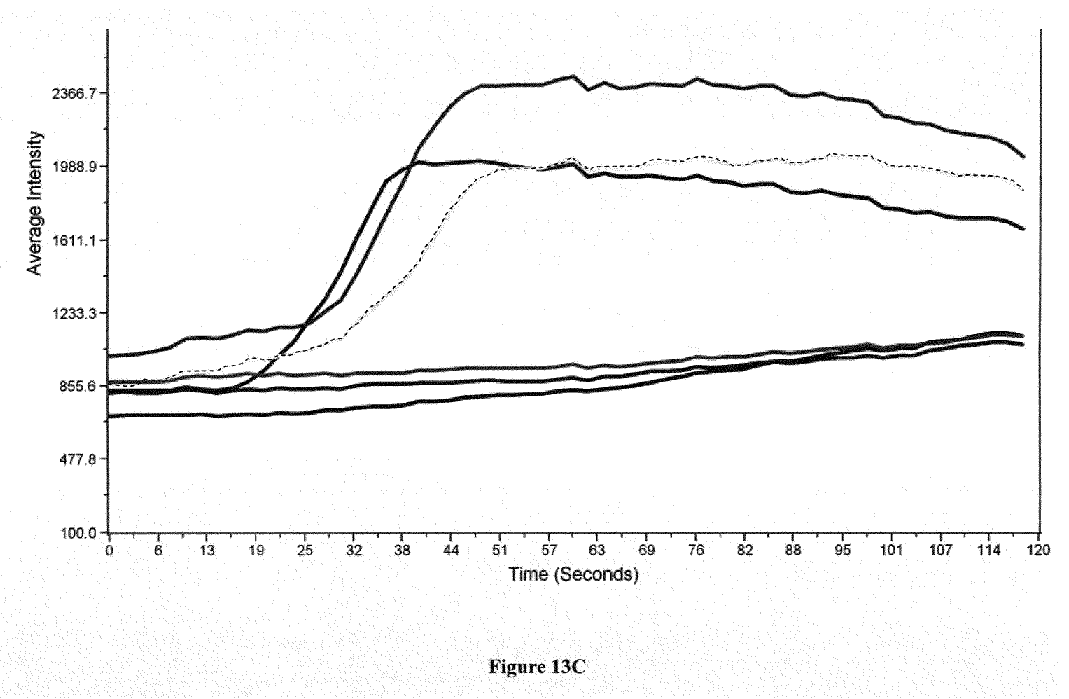

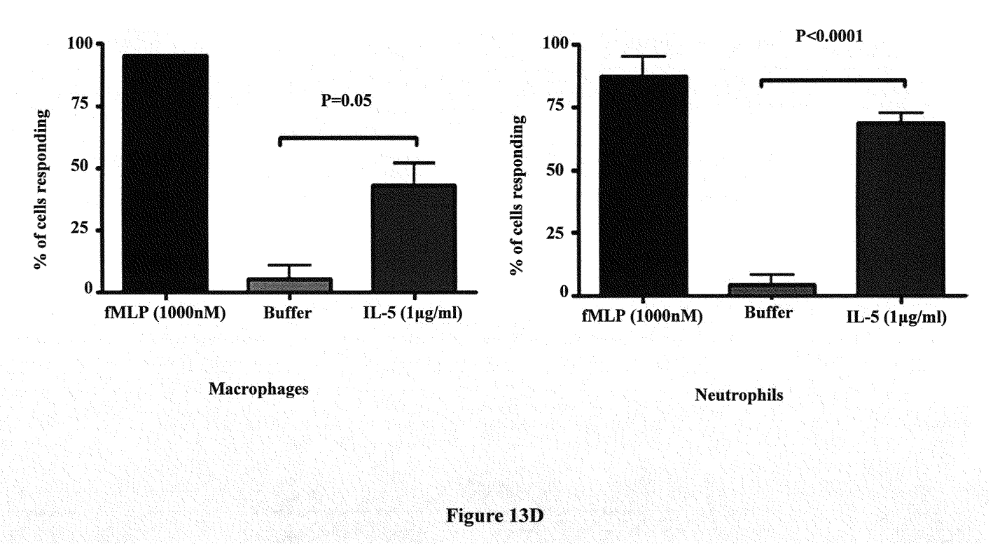

Treatment of either IFN-α or LPS primed macrophages with recombinant murine IL-5 (100 ng/ml), resulted in a significant increase in STAT1 nuclear translocation, an established downstream effect of IL-5Rα activation in eosinophils (FIG. 13A) (Pazdrak et al. (1995) J. Immunol., 155:397-402). Similar results were obtained for thioglycollate elicited peritoneal PMNs which also expressed high levels of IL-5Rα. The increase in transcriptional activation was associated with a dose dependent increase in IL-6 and IL-12p40 production (FIG. 13B). It was also demonstrated that IL-5 dramatically increases intracellular calcium mobilization in PMNs and macrophages (FIGS. 13C and 13D), consistent with its known chemotactic activity in EOS. Together, these data strongly indicate IL-5Rα is expressed on innate immune effector cells (PMNs and macrophages) in CLP and this receptor is biologically functional as determined by transcriptional activation, calcium signaling, and cytokine production.

### EXAMPLE 4

### Expression of IL-5 in Human Sepsis

As part of an ongoing study, whole blood, plasma and serum are being collected on healthy controls, subjects admitted to the ICU for non-septic etiologies (ICU controls) and subjects with sepsis and septic shock. Blood is collected on days 1, 3, 7, 14 and patients are followed clinically until hospital discharge and/or death. Similar to prior reports, no septic subject had detectable circulating eosinophils on presentation to the ICU, confirming that sepsis is an eosinopenic state (Weiner et al. (1952) Am. J. Med., 13:58-72; Venet et al. (2004) Clin. Immunol., 113:278-84).

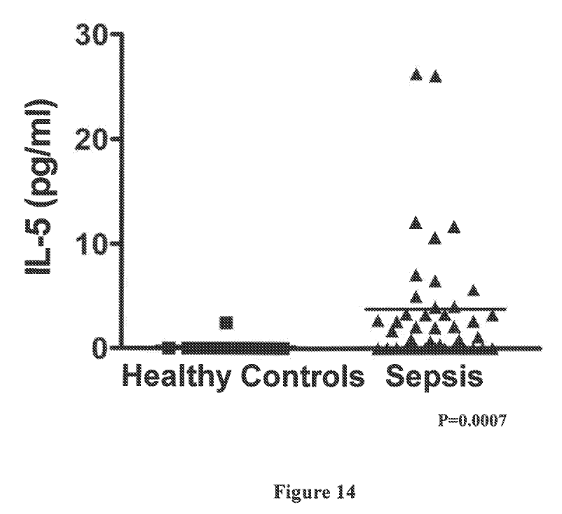

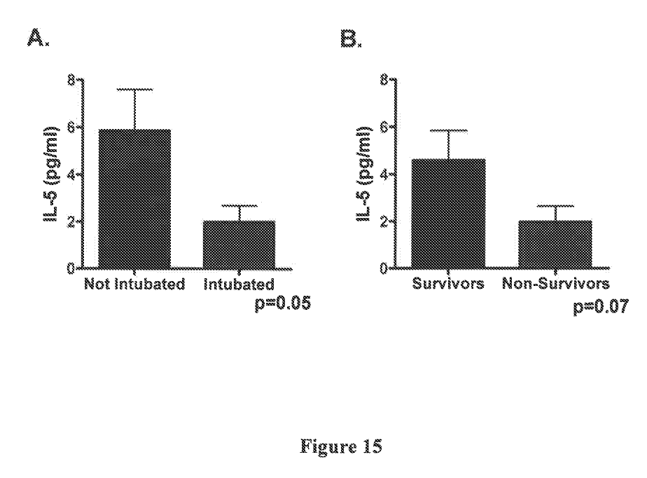

Levels of IL-5 were mildly increased in septic patients compared to healthy controls (p=0.0007) (FIG. 14). Interestingly, similar to the above murine data, levels of IL-5 were significantly higher in nonintubated compared to intubated subjects (FIG. 15A). A similar trend was observed in regards to mortality with survivors having a 2-fold higher level of IL-5 compared to non-survivors (FIG. 15B). Indeed, among septic individuals, those requiring mechanical ventilation have a trend towards lower IL-5 levels, compared to those who did not (5.433 vs. 2.146 pg/ml; p=0.07) with a similar trend observed for those who died compared to survivors (1.97 vs. 4.59 pg/ml; p=0.07). There was no association of IL-5 levels and the presence of bacteremia or acute renal failure, and there was no correlation between levels of IL-5 and either APACHE II or SAPS II score.

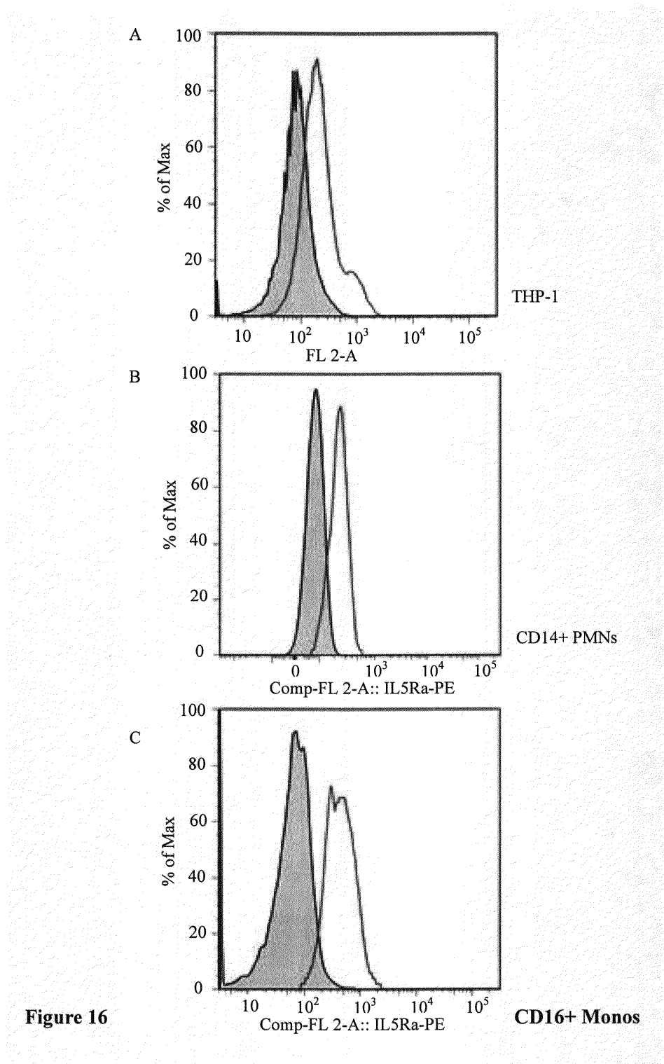

Finally, IL-5Rα expression was confirmed on human non-eosinophil granulocytes. Similar to the observations in murine cells, PMA differentiated THP-1 cells (a human monocytic cell line), express IL-5Rα after stimulation with LPS for 48 hours (FIG. 16A). While healthy humans have no detectable IL-5Rα on circulating monocytes and PMNs, 6/8 septic patients exhibited expression of IL-5Rα on CD16⁺ monocytes and CD14⁺ PMNs (FIGS. 16B and 16C). None of these patients had CCR3⁺granulocytes, confirming that sepsis is an eosinopenic state. The presence of the IL-5Rα on human PMNs was also confirmed by immunoblot. Together, these data indicate human innate immune effector cells can be modulated by exogenous IL-5 and high levels of IL-5 is associated with improved outcome in sepsis. Indeed, the confirmation of the IL-5Rα expression on PMNs and monocytes from septic patients and the ability to model this in vitro with THP-1 cells, establishes that this is not a species specific phenomenon and further indicates that IL-5Rα stimulation (e.g., via IL-5) to modulate the host innate immune response to sepsis.

### EXAMPLE 5

### Materials and Methods for Examples 1-4

Mice: WT C57BL/6 mice (Strain #00664) were obtained from Jackson Labs (Bar Harbor, Me.).

Reagents: All ELISAs were purchased from R&D Systems (Minneapolis, Minn.) and performed according to manufacturer's specifications. All antibodies for flow cytometry were purchased from BD Pharmingen (Franklin Lakes, N.J.).

Methods: CLP was performed as previously described. Briefly mice were anesthetized with 2.5% isofluroane and underwent CLP with specified needle gauge. Mice received 1 cc (5 cc/100 g) saline for resuscitation. For survival, mice received an additional 0.5 cc at 24 hours. Methods for CLP, murine sample collection, ELISAs, FACS for both murine and human cells NP-40 lysis, preparation of nuclear and cytoplasmic extracts for STAT-1 have been previously reported (Venet et al. (2004) Clin. Immunol., 113:278-84; Bass, D. A. (1975) J. Clin. Invest., 55:1229-36; Caldenhoven et al. (1999) Mol. Cell Biol. Res. Commun., 1:95-101; Stomski et al. (1999) Blood 94:1933-42; Liva et al. (2001) Neurochem. Res., 26:629-37; Pazdrak et al. (1995) J. Immunol., 155:397-402; Lee et al. (1997) J. Immunol., 158:1332-44; Foster et al. (1996) J. Exp. Med., 183:195-201). For experiments with recombinant IL-5, IL-5 (1 μg) or saline (200 μl) was administered at the specified time point.

Eosinophil isolation: Whole blood was collected from NJ1638^{±} mice, diluted into 70 ml PBS+2% FCS, and subjected to density-gradient centrifugation using percoll with a density of 1.084 g/ml at 2000 RPM for 45 minutes at 4° C. Interface cells were removed and washed with PBS+2% FCS. After red blood cells lysis, eosinophils were further isolated using Vantage cell sorter (Becton Dickinson) based on size and granularity, and resuspended in PBS+6% FCS. Population was >95% pure based on Diff-quick and CCR3⁺ staining on flow cytometry. Granules were sonicated on ice and spun at 3000 g×30 minutes to pellet out granule membrane fragments. The resulting supernatant was aliquoted and frozen at −80° C. till use.

Ex vivo EOS killing assay: Purified eosinophils or eosinophil granule proteins were resuspended to a concentration of 10⁶/ml in RPMI, and 100 μl was placed in wells of a 96-well plate. P. aeruginosa was grown shaking in LB broth at 37° C. until an OD of 1.0 was reached, or a concentration of 10⁹ CFU/ml. P. aeruginosa was resuspended to a concentration of 10⁷ CFU/ml in RPMI, and 100 μl was added to the wells of a 96-well plate, either with or without eosinophils. Plates were incubated for one hour at 37° C. and suspensions removed and centrifuged. Cell pellets were resuspended in 100 μl PBS, diluted in PBS, plated on LB agar and incubated at 37° C. overnight. Viable cell counts were made on the cultured plates.

Eosinophil adoptive transfer and in vivo infection: Purified eosinophils were resuspended to a concentration of 10⁶/ml and 200 μl eosinophils or saline control was injected into C57BL/6 mice one hour prior to infection with 10⁷ CFU Pseudomonas aeruginosa. Peritoneal lavage and blood samples were taken at 18 hours post infection and cultured on LB agar overnight at 37° C. for viable cell counts. For CLP experiments, eosinophils were injected 4 hours prior to surgery.

Flow Cytometry: Flow cytometry was performed as previously described (Pazdrak et al. (1995) J. Immunol., 155:397-402). Briefly, whole blood or peritoneal lavage were collected and 1×10⁶ cells were incubated with 100 μl Fc block for 15 minutes then stained with appropriate antibody (αCD125, αCCR3, αCD4, αCD8, αLY6g, αF4/80) at optimal concentration for 45 minutes in dark. Red cells were lysed with RBC lysis buffer, fixed with 0.1% paraformaldehyde and run on a BD LSRII 8-color analyzer and data analyzed with FloJo software (Tree Star, Ashland, Oreg.). All reagents are from BDPharmigen. Compensation with BD compensation beads was performed before each use. PMNs were identified by FSC/SSC characteristics as Ly6G⁺, eosinophils via FSC/SSC and CCR3⁺, mononuclear cells by FSC/SSC, F4/80 and CD14⁺, T-cells by FSC/SSC and further subgrouped by CD4⁺ and CD8⁺ staining. Isotype labeled cells were used to control for background staining.

Cell Culture: RAW 264.7 or thioglycollate elicited PM were grown in RPMI+10% FCS at a density of 5×10⁵ cells/ml in 6 well dishes. Cells were incubated with designated reagents for specified time points. Cells were collected and nuclear and cytoplasmic extracts made as previously described (Gold et al. (2003) Infect. Immun., 71:3521-8). Immunoblots were performed as previously described (Hoshino et al. (2004) J. Immunol., 172:6251-8; Gold et al. (2004) Infect. Immun., 72:645-50; Gold et al. (2007) PLoS ONE 2:e736; Hoshino et al. (2007) J. Infect. Dis., 195:1303-10).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating sepsis or reducing the risk of developing sepsis in a patient in need thereof comprising administering a composition comprising IL-5 and a pharmaceutically acceptable carrier.

2. The method of claim 1, wherein said method further comprises the administration of at least one other anti-sepsis agent.

3. The method of claim 2, wherein said anti-sepsis agent is selected from the group consisting of activated protein C, anti-thrombin III, bactericidal/permeability-increasing protein (BPI), anti-infectives, and antibiotics.

4. The method of claim 3, wherein said antibiotic is selected from the group consisting of β-lactam, penicillin, cephalosporin, vancomycin, bacitracin, macrolide, erythromycin, lincosamide, clindomycin, chloramphenicol, tetracycline, immunocycline, chlortetracycline, oxytetracycline, demeclocycline, methacycline, doxycycline, minocycline, aminoglycoside, gentamicin, amikacin, neomycin, amphotericin, cefazolin, clindamycin, mupirocin, sulfonamide, trimethoprim, rifampicin, metronidazole, quinolone, novobiocin, polymixin, gramicidin, imipenem, meropenem, cefoperazone, cefepime, nafcillin, linezolid, aztreonam, piperacillin, tazobactam, ampicillin, sulbactam, clindamycin, metronidazole, levofloxacin, carbapenem, linezolid, rifampin, and metronidazole.

5. The method of claim 1, wherein said composition is administered intravenously.

6. The method of claim 1, wherein said composition is administered by an osmotic infusion pump.

7. The method of claim 1, further comprising monitoring said patient for the presence of sepsis or the severity of sepsis.

8. The method of claim 1, wherein said administration of the composition comprising IL-5 increases the concentration of neutrophils at the site of infection.
